(19) United States
(12) Patent Application Publication
(10) Pub. No.: US 2023/0174456 A1
(43) Pub. Date: Jun. 8, 2023

(54) PROCESS FOR PRODUCING HYDROXYALKYL (METH)ACRYLATE ESTERS BY OXIDATIVE CLEAVAGE OF METHACROLEIN ACETALS

(71) Applicant: Röhm GmbH, Darmstadt (DE)

(72) Inventors: Andreas RÜHLING, Darmstadt (DE); Steffen Krill, Muehltal (DE); Florian Zschunke, Frankfurt (DE); Belaid Ait Aissa, Darmstadt (DE)

(73) Assignee: Röhm GmbH, Darmstadt (DE)

(21) Appl. No.: 17/997,168

(22) PCT Filed: Apr. 19, 2021
(86) PCT No.: PCT/EP2021/060020

§ 371 Date: Oct. 26, 2022

(30) Foreign Application Priority Data
Apr. 30, 2020 (EP) 20172256.8

(51) Int. Cl. C07C 69/54; C07C 67/39

(52) U.S. Cl. CPC C07C 69/54; C07C 67/39

(57) ABSTRACT

A process can be used for producing hydroxyalkyl (meth)acrylate esters, in particular hydroxyethyl methacrylate (HEMA). The process involves a first reaction of (meth)acrolein with at least one polyhydric alcohol, in particular ethylene glycol, in the presence of a first catalyst C1, wherein a first reaction product containing a cyclic acetal is obtained. The process then involves a second reaction of the first reaction product with oxygen in the presence of a second catalyst C2, wherein a second reaction product containing at least one hydroxyalkyl (meth)acrylate ester is obtained. After the first reaction, water and optionally further components, in particular (meth)acrolein and/or the polyhydric alcohol, e.g. ethylene glycol, are at least partially removed from the first reaction product.

The present invention relates to a process for producing hydroxyalkyl (meth)acrylate esters, in particular hydroxyethyl methacrylate (HEMA), comprising, in a first reaction step, the reaction of (meth)acrolein with at least one polyhydric alcohol, in particular ethylene glycol, in the presence of a first catalyst C1, wherein a first reaction product comprising a cyclic acetal is obtained, and, in a second reaction step, the reaction of the first reaction product with oxygen in the presence of a second catalyst C2, wherein a second reaction product comprising at least one hydroxyalkyl (meth)acrylate ester is obtained, wherein, after the first reaction step, water and optionally further components, in particular (meth)acrolein and/or the polyhydric alcohol, e.g. ethylene glycol, are at least partially removed from the first reaction product.

Hydroxyalkyl esters based on methacrylic acid and/or acrylic acid are industrially important monomers or comonomers for the production of polymethyl(meth)acrylates. Hydroxyalkyl (meth)acrylate esters and polymers produced therefrom are employed for various uses, for example for paints, adhesives, contact lenses, polymer crosslinkers and materials for 3D printing. Of particular industrial importance is hydroxyethyl methacrylate (HEMA).

### PRIOR ART

The production of HEMA starting from methacrylic acid and ethylene oxide using chromium catalysts is frequently described in the prior art, e.g. in WO 2012/116870 A1, JP 5 089 964 B2 and US 2015/01267670. Often, a stabilizer is added to the very reactive hydroxyalkyl (meth)acrylate ester monomer (e.g. in EP-B 1 125 919).

In addition to the production of HEMA starting from methacrylic acid, the production of other hydroxyalkyl (meth)acrylate esters using for example propylene oxide or other substituted oxiranes is also described in the prior art, e.g. in JP 2008143814, JP 2008127302. The reaction of (meth)acrylic acid with the corresponding oxiranes is usually carried out with homogeneous catalysts, wherein some or all of the methacrylic acid is initially charged in the presence of the dissolved catalyst and a stabilizer and the gaseous or liquid oxirane is metered in. Besides the metering-in, the mixing of the reactants and of the catalyst is also important in order that the desired hydroxyalkyl (meth)acrylate ester is obtained in these reactions in high, constant product quality and that the production equipment can be operated continuously and be easily cleaned. An embodiment suitable for this purpose is for example described in WO 2012/1168770 A1, wherein the reactor is fitted with an injector-mixer nozzle and a circulation line. Towards the end of the reaction, the desired hydroxyalkyl (meth)acrylate esters are typically present in high concentration, whereas the starting material methacrylic acid is substantially depleted or no longer present.

The subsequent workup and isolation of the hydroxyalkyl (meth)acrylate esters is often effected by distillation and can be operated continuously (e.g. as described in DE 10 2007 056926 A1 and EP 1090904 A2).

Commercial products generally undergo purification by distillation to a purity of greater than 97%, while for special uses the acid-based secondary components and crosslinkers are reduced further. The content of hydroxyalkyl (meth)acrylate esters is in this case usually over 99%. Such purification takes place in a plurality of distillation steps and is described for example in EP 2427421 B1. Irrespective of the desired product quality, it is often necessary to add at least one stabilizer during purification. The within-specification product can for example contain hydroquinone monomethyl ether and 4-hydroxy-2,2,6,6-tetramethylpiperidine-N-oxyl (TEMPOL) or else combinations of a plurality of stabilizers (e.g. as described in WO 2010/105894 A2). Product compositions are also described that, in addition to stabilizers, also contain reaction by-products such as hydroxyethyl acetate and should have particular storage stability (e.g. as described in EP 1125919 A2).

The catalysts used in the reaction are often based on chromium salts, as described e.g. in patent documents WO 2012/116870 A1, JP 5 089 964 B2 and US 2015/01267670. In addition to chromium catalysts, catalysts based on iron salts, are also described, e.g. in U.S. Pat. No. 4,365,081. A variety of other metal-containing, soluble catalysts from the group of transition metals with a wide range of counterions or ligands is described in the literature.

A problem in these known and customary production processes is the use of ethylene oxide, which is a highly toxic, carcinogenic, highly flammable and explosive compound. Such processes accordingly place high demands on handling, transport, storage, etc. of ethylene oxide. The transport and use of ethylene oxide are in the EU and other countries such as the USA strongly regulated and therefore costly. Information concerning the USA is described for example in the EPA Clean Air Act. A tightening of the conditions for handling ethylene oxide in the USA is likely, based on the upcoming 2020 update of the National Emission Standards for Hazardous Air Pollutants (NESHAP).

The expansion of capacities for the production of HEMA is accordingly often limited by local availability and by the regulation of ethylene oxide (e.g. EPA Clean Air Act for USA).

In the known processes, this means that the use of ethylene oxide must in particular be critically assessed when ethylene oxide is present in excess or present in static, relatively high concentrations. This is the case in most of the processes described in the prior art. Increased safety precautions must therefore be taken, in particular the admixing and static concentration of ethylene oxide must be strictly regulated via the setting of metering times and metering rates and by monitoring the temperature and dissipating the heat produced in the reaction.

A further disadvantage of the production processes of the prior art is the use of toxic and carcinogenic chromium salts, often in oxidation state +VI. The first chromium salts of other oxidation states have already been classified as critical in respect of their use and disposal (see for example file No. WD5-3000-044/19 of the German Bundestag and REACH Annex XVII on chromium +VI compounds). Further bans, requirements and regulations for stricter occupational safety can be expected to follow in the future, which will result at least in additional costs. Residues of chromium salts arise in all established processes, particularly during processing as distillation bottoms residues.

Such bottoms residues need to be disposed of laboriously and carefully, in order to ensure there is no escape of chromium into the environment. In summary, this gives rise to disadvantages in the established processes with relation to high costs for the use of catalysts and the resulting disposal of catalyst residues as well as high capital expenditure on production equipment and high ongoing operating costs due to long reaction times.

EP 0 704 441 A2 describes a process for producing 2-vinyl-1,3-dioxolane by the reaction of acrolein with ethylene glycol in the presence of a solid acid catalyst. A particular point to note here is that the reaction has higher selectivity at low temperatures of below 20° C. At higher temperature, an increased formation of by-products through addition of the alcohol at the 4-position of acrolein in the sense of a Michael-type reaction is described. When using polyhydric alcohols, the formation of oligomeric and polymeric compounds must moreover be expected, which would make reaction control and purification more difficult. This publication does not make any clear statements thereon.

EP 0 485 785 A1 describes a process for producing alpha,beta-unsaturated acetals, in particular starting from methacrolein, with methanol used as the alcohol. In this process, methanol and methacrolein are separated from the acetal by distillation and brought to reaction at room temperature. Unreacted starting material runs together with the acetal into the bottoms receiver of the column. The process described here is not however applicable for dihydric alcohols such as ethylene glycol, since the dihydric alcohol has a higher boiling point than the corresponding dioxolane. For such a reaction, large amounts of by-products would accordingly be expected by analogy with the description in EP 0 704 441 A2.

The document JPH11315075A describes the reaction of methacrolein with ethylene glycol using a heterogeneous catalyst, e.g. a zeolite or mixtures of silicon dioxide and aluminium oxide, and an azeotropic entrainer such as cyclohexane or toluene. Only batch processes are described therein, which must be operated at high temperatures because of the nature of the chosen entrainer.

The Japanese patent application JP 43-11205 describes the oxidative cleavage of a cyclic acetal to afford an unsaturated acrylate-based hydroxy ester. The oxidative cleavage of a methacrolein-based cyclic acetal affording 2-hydroxyethyl methacrylate is likewise described. However, a conversion of only 25% is obtained here within 6 hours, which based on the space-time yield must be considered inadequate and not very economic. The document does not describe the production of the cyclic acetals.

The Japanese patent application JP 2009 274987 describes a process for producing hydroxyalkyl (meth)acrylates, e.g. hydroxyethyl (meth)acrylate or hydroxypropyl (meth)acrylate, by oxidation of a cyclic acetal in the presence of a special heterogeneous noble metal-containing catalyst. However, the long reaction times in particular make the processes described here unsuitable for industrial use, particularly in a continuous process.

The chemoselective oxidation of aromatic acetals by oxygen with the formation of hydroxyalkyl esters using a palladium catalyst is moreover described in Sawama et al. (Org. Lett. 2016, 18, 5604). The reaction of non-aromatic substrates, and of alpha,beta-unsaturated compounds in particular, is not described here. Sawama et al. report methanol or ethylene glycol to be the best solvent.

The document U.S. Pat. No. 9,593,064 B2 (S. S. Stahl et al.) describes palladium catalysts on activated carbon augmented by two dopants, for example bismuth and tellurium. The catalysts are used for the production of esters by direct oxidative esterification of organic alcohols in the presence of methanol or ethanol. Cyclic acetals as substrates are not described.

In consideration of the prior art, it is therefore an object of the present invention to provide a process for producing hydroxyalkyl (meth)acrylate esters, in particular hydroxyethyl methacrylate (HEMA), wherein ethylene oxide and optionally also methacrylic acid are replaced by different reactants that are safe, inexpensive and have good global availability. In addition, the production process should in particular be competitive with known processes of the prior art and not be subject to the above-described disadvantages of conventional processes.

A further object of the present invention is to provide a hydroxyalkyl (meth)acrylate ester product that meets the usual requirements for purity and content of secondary components or that can be purified further with the least possible outlay so that it meets the appropriate requirements. In particular, a hydroxyalkyl (meth)acrylate ester product should be provided that has the lowest possible content of crosslinking by-products (compounds having two or more C═C double bonds), e.g. ethylene dimethacrylate.

It was surprisingly found that the objects described above were achieved by the process according to the invention. In particular, it was found that methacrolein and ethylene glycol can be used as reactants in the production of hydroxyethyl methacrylate (HEMA), wherein initially, with the elimination of water, a cyclic acetal is formed that then undergoes catalytic oxidation with oxygen, selectively affording HEMA. Ethylene glycol and other polyhydric alcohols are typically inexpensive and available worldwide. The production of methacrolein from propionaldehyde and formaldehyde is known to those skilled in the art and is practised on an industrial scale. Methacrolein can additionally be obtained by oxidation of isobutene or starting from t-butanol. Processes for the production of (meth)acrolein are described for example in Ullmann's Encyclopedia of Industrial Chemistry, 2012, Wiley-VCH Verlag GmbH, Weinheim (DOI: 10.1002/14356007.a01_149.pub2).

### DESCRIPTION OF THE INVENTION

The present invention relates to a process for producing hydroxyalkyl (meth)acrylate esters, comprising the steps of:
a. reacting (meth)acrolein with at least one polyhydric alcohol in the presence of a first catalyst C1, wherein a first reaction product comprising at least one cyclic acetal is obtained;
b. at least partially removing water from the first reaction product;
c. reacting the first reaction product with oxygen in the presence of a second catalyst C2, wherein a second reaction product comprising at least one hydroxyalkyl (meth)acrylate ester is obtained.

In a preferred embodiment, the process for producing hydroxyalkyl (meth)acrylate esters is a continuous or semicontinuous process, preferably a continuous process.

The expressions “(meth)acrylate” and “(meth)acrylic ester” encompass for the purposes of the invention acrylate and/or methacrylate. The expression “(meth)acrolein” correspondingly encompasses for the purposes of the invention acrolein and/or methacrolein.

In a preferred embodiment, ethylene glycol is used as the polyhydric alcohol. The hydroxyalkyl (meth)acrylate ester is preferably hydroxyethyl methacrylate ester (HEMA) and the cyclic acetal is in particular methacrolein ethylene glycol acetal (2-isopropenyl-1,3-dioxolane).

The invention additionally encompasses the use of alkyl-substituted glycols as the polyhydric alcohol instead of ethylene glycol. In particular, the invention relates likewise to the production of hydroxypropyl (meth)acrylates, in particular 2-hydroxypropyl (meth)acrylate (HPMA), wherein propanediol, e.g. 1,2-propanediol, is used in particular as the polyhydric alcohol.

Step a—Reaction to the Cyclic Acetal

The process according to the invention comprises in step a the reaction of (meth)acrolein with at least one polyhydric alcohol in the presence of a first catalyst C1, wherein a first reaction product comprising at least one cyclic acetal is obtained. Typically, water is obtained as a further product of acetal formation.

For the purposes of the present invention, a polyhydric alcohol is a compound, more particularly an organic compound, that contains two or more hydroxy (—OH) groups. The polyhydric alcohol is preferably at least one alcohol containing 2 to 10 carbon atoms, preferably 2 to 5 carbon atoms, more preferably 2 to 3 carbon atoms, and containing two or more hydroxy groups, preferably two or three hydroxy groups, more preferably two hydroxy groups. The hydroxy groups can preferably be present in the 1,2-, 1,3- or 1-4-positions in the structure of the polyhydric alcohol, e.g. in the diol. The hydroxy groups are more preferably present in the 1,2-position in the polyhydric alcohol. The polyhydric alcohol may additionally contain further functional groups, for example alkoxy groups, aryl groups, phosphonate groups, phosphate groups, alkenyl groups, alkynyl groups, masked carbonyl groups or ester units.

The at least one polyhydric alcohol is particularly preferably selected from ethylene glycol, propylene glycol, butanediol and/or glycerol. In a preferred embodiment, the at least one polyhydric alcohol is ethylene glycol. In a preferred embodiment, a polyhydric alcohol as described above is exclusively used.

The cyclic acetal preferably has a structure as shown in formula (I):

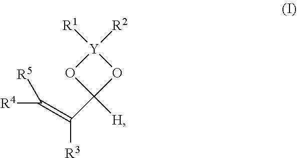

where Y is a C₂-C₁₀ alkylene group, preferably a C₂-C₄ alkylene group, more preferably a C₂-C₃ alkylene group; R¹ and R² are independently selected from H, C₁-C₂₀ alkyl, C₁-C₂₀ hydroxyalkyl, C₁-C₂₀ alkoxy and C₆-C₂₀ aryl; R³ is H or C₁-C₂₀ alkyl, preferably H or methyl; R⁴ and R⁵ are independently selected from H, C₁-C₂₀ alkyl, C₁-C₂₀ hydroxyalkyl and C₆-C₂₀ aryl. R¹ and R² are preferably independently selected from H, C₁-C₆ alkyl, C₁-C₆ hydroxyalkyl and C₆-C₁₂ aryl. R⁴ and R⁵ are preferably independently selected from H, C₁-C₆ alkyl, C₁-C₆ hydroxyalkyl and C₆-C₁₂ aryl, more preferably from H and C₁-C₆ alkyl. R¹ and R⁵ are particularly preferably H. R³ is particularly preferably methyl.

The cyclic acetal particularly preferably has a structure as shown in formula (11):

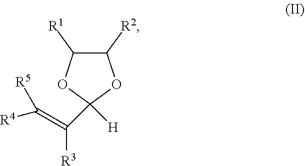

where R¹, R², R³, R⁴ and R⁵ are as defined above.

The reaction in step a of (meth)acrolein with at least one polyhydric alcohol to form the cyclic acetal is typically an equilibrium reaction wherein, in particular, the conversion is 10 to 75%, preferably 15 to 50%. In a continuous process, the conversion typically relates to the conversion per pass in step a.

The reaction in step a is preferably carried out in the presence of at least one acidic compound as catalyst C1, selected in particular from Brønsted acids and Lewis acids. The at least one catalyst C1 is preferably selected from the group consisting of phosphoric acid, sulfuric acid, sulfonic acids, carboxylic acids (e.g. formic acid, acetic acid), lanthanoid salts, metal salts of elements of groups 3 to 15 of the periodic table and ion-exchange resins containing at least one acidic group selected from phosphonic acid (—P(═O)(OH)₂), sulfonic acids (—S(═O)₂OH) and carboxylic acids (—COOH) (e.g. acetic acid (—CH₂—CH₂—C(═O)OH)). The at least one catalyst C1 is particularly preferably selected from the group consisting of phosphoric acid, sulfuric acid, sulfonic acids, carboxylic acids (e.g. formic acid, acetic acid) and ion-exchange resins containing at least one acidic group selected from sulfonic acids (—S(═O)₂OH) and carboxylic acids (—COOH).

It is additionally possible to use at least one Lewis acid as catalyst C1, selected for example from lanthanoid salts and metal salts of elements of groups 3 to 15 of the periodic table, in particular the compounds may be halides, hydroxides, mesylates, triflates, carboxylates and/or chalcogenides. For example, at least one Lewis acid can be used as catalyst C1 selected from halides, hydroxides, mesylates, triflates, carboxylates and chalcogenides (preferably selected from halides) of alkali metals (in particular Li⁺, Na⁺, K⁺), alkaline earth metals (in particular Be²⁺, Mg²⁺, Ca²⁺), B³⁺, Al³⁺, In³⁺, Sn²⁺, Sn⁴⁺, Si⁴⁺, Sc³⁺, Ti⁴⁺, Pd²⁺, Ag⁺, Cd²⁺, Pt²⁺, Au⁺, Hg²⁺, In³⁺, Tl³⁺, Pb²⁺; and from organic salts, e.g. alkoxides, of Ti⁴⁺, Sn⁴⁺ and B³⁺, e.g. Ti(OH)₄, B(OR)₃, Sn(OR)₄, where R is C₁-C₁₀ alkyl. For example, the catalyst C1 used may preferably be a known Lewis acid selected from BCl₃, BF₃, B(OH)₃, B(CH₃)₃, AlCl₃, AlF₃, TiCl₄, Ti(OH)₄, ZnCl₂, SiBr₄, SiF₄, PF₅, Ti(OR)₄, B(OR)₃ and Sn(OR)₄, where R is C₁-C₁₀ alkyl.

The catalyst C1 is preferably one or more Brønsted acids that preferably has a pKa of less than or equal to 5, more preferably of less than or equal to 2.

The catalyst C1 preferably contains at least one acidic group that has a pKa of less than or equal to 5, more preferably of less than or equal to 2.

In a preferred embodiment, the reaction in step a is carried out in the presence of at least one acidic compound as catalyst C1, selected from Brønsted acids and Lewis acids, wherein the catalyst is present in heterogeneous or homogeneous form.

The catalyst C1 is further preferably a heterogeneous catalyst. For example, the catalyst may be supported on a polymer matrix, composite matrix and/or oxidic support material. Likewise preferred is the use of heterogeneous polyacids, for example ones based on molybdates. A particular advantage of using a heterogeneous catalyst C1 is that the catalyst C1 and the reaction mixture can be separated from one another more easily and/or that the catalyst C1 can be reused (and generally operated) for a longer period.

In a preferred embodiment, the reaction of (meth)acrolein with the at least one polyhydric alcohol in step a is carried out with a molar ratio of (meth)acrolein to polyhydric alcohol(s) within a range from 1:50 to 50:1, preferably from 1:10 to 10:1, more preferably from 1:3 to 3:1.

The reaction of (meth)acrolein with the polyhydric alcohol in step a is preferably carried out within a temperature range from −50° C. to 100° C., more preferably from −10° C. to 30° C., particularly preferably 0 to 20° C. The reaction of (meth)acrolein with the polyhydric alcohol in step a is preferably carried out within a pressure range from 0.5 to 10 bar (absolute), more preferably from 1 to 5 bar (absolute). In particular, the reaction in step a is carried out at a temperature and/or at a pressure within the ranges indicated above.

In a preferred embodiment, the reaction of (meth)acrolein with the polyhydric alcohol (for example ethylene glycol) in step a can be performed in the presence of a solvent. A solvent typically selected from linear or cyclic alkanes (e.g. hexane, octane, cyclohexane), aromatic hydrocarbons (e.g. toluene, benzene), halogenated hydrocarbons (e.g. chloroform, carbon tetrachloride, hexachloroethane, hexachlorocyclohexane, chlorobenzene), alcohols (e.g. methanol, ethanol, n-butanol, tert-butanol), ethers (e.g. diisopropyl ether, tetrahydrofuran, 1,3-dioxane) or mixtures thereof can be used. Further polar solvents that are chemically inert in the reaction according to step a are additionally known to those skilled in the art. The reaction in step a is typically carried out in a reaction mixture containing 1% to 90% by weight, preferably 5% to 50% by weight, based on the entire reaction mixture, of at least one solvent.

In a particularly preferred embodiment, the reaction in step a is carried out without solvent.

Step b—Removal of Water from the First Reaction Product

The process according to the invention comprises in step b the at least partial removal from the first reaction product of the water formed in the reaction. Step b of the process according to the invention preferably additionally comprises the at least partial removal of the unreacted polyhydric alcohol, e.g. ethylene glycol, and/or of the unreacted (meth)acrolein from the first reaction product obtained in step a.

The removal of water and optionally of polyhydric alcohol and optionally of (meth)acrolein is preferably effected by at least one distillation step (in particular in the form of a distillation column, e.g. columns 7 and 10).

In a preferred embodiment, step b comprises the removal of unreacted (meth)acrolein and/or unreacted polyhydric alcohol, typically together with the water, from the first reaction product, optional separation from the water, and the recycling thereof to the reaction in step a.

Step b particularly preferably comprises the removal in a first separation step, preferably in a distillation step, of (meth)acrolein and of at least some of the water from the first reaction product. In particular, (meth)acrolein and its azeotrope with water are in this first separation step removed from the first reaction product by distillation. In particular, the first separation step is carried out in a distillation column (e.g. column 7).

In addition, step b preferably comprises the removal, in at least two separation steps, preferably two distillation steps, of water, (meth)acrolein and polyhydric alcohol from the first reaction product comprising at least one cyclic acetal.

Step b particularly preferably comprises the at least partial removal (e.g. in a second separation step) of the polyhydric alcohol and optionally of water and optionally of high boilers from the reaction product, preferably in a distillation step (e.g. column 10).

In a preferred embodiment, step b comprises the removal, in at least two separation steps, of water, unreacted (meth)acrolein and unreacted polyhydric alcohol from the first reaction product comprising at least one cyclic acetal, wherein, in a first separation step, preferably in a distillation step (e.g. column 7), (meth)acrolein and at least some of the water (e.g. (meth)acrolein and its azeotrope with water) are removed from the first reaction product, and wherein, in a second separation step, preferably in a distillation step (e.g. column 10), the polyhydric alcohol and optionally water and optionally high boilers are at least partially removed from the first reaction product.

The removal of the (meth)acrolein in step b is preferably carried out in a manner such that the content of (meth)acrolein in the reaction mixture in step c is less than 10% by weight, preferably less than 8% by weight, particularly preferably less than 5% by weight, based on the total reaction mixture in step b.

The removal of water in step b is preferably carried out in a manner such that the content of water in the reaction mixture in step c is less than 5% by weight, preferably less than 2% by weight, particularly preferably less than 1% by weight, based on the total reaction mixture in step b.

The removal of the polyhydric alcohol in step b is preferably carried out in a manner such that the content of the polyhydric alcohol in the reaction mixture in step c is less than 10% by weight, preferably less than 8% by weight, particularly preferably less than 5% by weight, based on the total reaction mixture in step c. Particularly preferably, the polyhydric alcohol is in step b almost completely removed from the first reaction product.

Step c—Oxidation

The process according to the invention comprises in step c the reaction of the first reaction product, comprising at least one cyclic acetal (in particular 2-isopropenyl-1,3-dioxolane), with oxygen in the presence of a second catalyst C2, wherein a second reaction product comprising at least one hydroxyalkyl (meth)acrylate ester is obtained.

Step c comprises typically an oxidative esterification of the cyclic acetal obtained in reaction step a. Typically, the hydroxyalkyl (meth)acrylate ester is obtained by oxidative ring-opening of the cyclic acetal.

The hydroxyalkyl (meth)acrylate ester preferably has a structure as shown in formula (III):

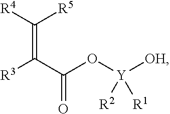

where Y is a C₂-C₁₀ alkylene group, preferably a C₂-C₄ alkylene group, more preferably a C₂-C₃ alkylene group; R¹ and R² are independently selected from H, C₁-C₂₀ alkyl, C₁-C₂₀ hydroxyalkyl, C₁-C₂₀ alkoxy and C₆-C₂₀ aryl; R³ is H or C₁-C₂₀ alkyl, preferably H or methyl; R⁴ and R⁵ are independently selected from H, C₁-C₂₀ alkyl, C₁-C₂₀ hydroxyalkyl and C₆-C₂₀ aryl. R¹ and R² are preferably independently selected from H, C₁-C₆ alkyl, C₁-C₆ hydroxyalkyl and C₆-C₁₂ aryl. R⁴ and R⁵ are preferably independently selected from H, C₁-C₆ alkyl, C₁-C₆ hydroxyalkyl and C₆-C₁₂ aryl, more preferably from H and C₁-C₆ alkyl. R⁴ and R⁵ are particularly preferably H. R³ is particularly preferably methyl.

The hydroxyalkyl (meth)acrylate ester particularly preferably has a structure as shown in formula (IV):

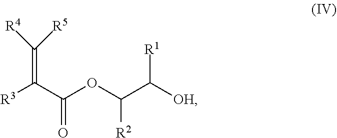

where R¹, R², R³, R⁴ and R⁵ are as defined above.

The reaction in step c is preferably carried out in the presence of a metal-containing and/or metalloid-containing heterogeneous catalyst system as catalyst C2, more preferably a noble metal-containing, heterogeneous catalyst system as catalyst C2.

For the purposes of the present invention, the term noble metal encompasses the elements ruthenium (Ru), rhodium (Rh), palladium (Pd), osmium (Os), iridium (Ir), platinum (Pt), silver (Ag), gold (Au), and rhenium (Re), in particular gold (Au), silver (Ag) and the platinum-group metals (Ru, Rh, Pd, Os, Ir, Pt).

The at least one catalyst C2 is preferably a heterogeneous catalyst comprising one or more support materials and one or more active components, wherein the support material is selected from activated charcoal, silicon dioxide, aluminium oxide, titanium dioxide, alkali metal oxides, alkaline earth metal oxides and mixtures thereof, and wherein the active component comprises at least one element selected from palladium (Pd), platinum (Pt), iridium (Ir), rhodium (Rh), ruthenium (Ru), gold (Au), cobalt (Co), nickel (Ni), zinc (Zn), copper (Cu), iron (Fe), selenium (Se), tellurium (Te), arsenic (As), antimony (Sb), bismuth (Bi), germanium (Ge), tin (Sn) and lead (Pb), wherein the elements can be present in elemental form, as an alloy, or in the form of compounds thereof in any desired oxidation state (preferably in the form of oxides thereof).

The support material is in particular silicon dioxide and/or aluminium oxide, preferably aluminium oxide.

The active component particularly preferably comprises at least one element selected from palladium (Pd), platinum (Pt), iridium (Ir), rhodium (Rh), ruthenium (Ru), gold (Au), bismuth (Bi) and/or tellurium (Te), wherein the elements can be present in elemental form, as an alloy, or in the form of compounds thereof in any desired oxidation state (preferably in the form of oxides thereof). The active component of the catalyst C2 particularly preferably includes palladium.

The at least one catalyst C2 is particularly preferably a heterogeneous catalyst comprising silicon dioxide and/or aluminium oxide as support material and comprising as active component at least one element selected from palladium, bismuth and tellurium, wherein the elements can be present in elemental form, as an alloy, or in the form of compounds thereof in any desired oxidation state (preferably in the form of oxides thereof).

In a preferred embodiment, the active components of the catalyst C2 is the combination of at least one noble metal selected in particular from gold (Au), silver (Ag), palladium (Pd), platinum (Pt), iridium (Ir), rhodium (Rh) and ruthenium (Ru); and at least one further element (dopant) selected in particular from selenium (Se), tellurium (Te), arsenic (As), antimony (Sb), bismuth (Bi), germanium (Ge), tin (Sn) and lead (Pb), wherein the elements can be present in elemental form, as an alloy, or in the form of compounds thereof in any desired oxidation state (preferably in the form of oxides thereof).

In a particularly preferred embodiment, the active components of the catalyst C2 is the combination of palladium (Pd) and at least one further element (dopant) selected from selenium (Se), tellurium (Te), antimony (Sb) and bismuth (Bi), wherein the elements can be present in elemental form, as an alloy, or in the form of compounds thereof in any desired oxidation state (preferably in the form of oxides thereof).

The at least one catalyst C2 is further preferably a heterogeneous catalyst comprising silicon dioxide and/or aluminium oxide as support material and an active component, wherein the active component is palladium and at least one further element (dopant) selected from selenium (Se), tellurium (Te) and bismuth (Bi), wherein the elements can be present in elemental form, as an alloy, or in the form of oxides thereof in any desired oxidation state.

The catalyst C2 preferably contains at least 70% by weight, preferably 70% to 99.9% by weight, based on the total catalyst mass, of one or more support materials and not more than 30% by weight, preferably 0.1% to 30% by weight, based on the total catalyst mass, of one or more active components.

In a preferred embodiment, the heterogeneous second catalyst C2 in step c is used in the form of a powder. The reaction in step c is here preferably carried out in the presence of a dispersed, pulverulent catalyst C2.

The amount of catalyst C2 in step c, based on the total mass of reaction mixture, is typically 0.1% to 30% by weight, preferably 1.0% to 20% by weight and more preferably 2.0% to 15% by weight.

In an alternative embodiment, step c is carried out in a fixed-bed or trickle-bed reactor, the ratio of catalyst to reaction mixture being expressed by the parameter LHSV (liquid hourly space velocity) that is known to those skilled in the art, e.g. in L liquid/(kg catalyst×hr) or in kg liquid/(kg catalyst×hr). The LHSV is typically 0.05 to 15, preferably between 0.1 to 10 and more preferably between 1 and 5.

In the reaction in step c, a reaction mixture comprising the first reaction product and the second catalyst C2 is preferably contacted with an oxygen-containing gas. This can be achieved in reactors or units in the reactor periphery known to those skilled in the art, such as bubble columns, gas-flushed stirred-tank reactors and trickle-bed reactors.

In the reaction in step c, the reaction mixture is typically contacted with an oxygen-containing gas, wherein an oxygen-containing offgas is obtained. The oxygen-containing offgas typically has an oxygen content within the range from 1% to 10% by volume, preferably 1% to 5% by volume, based on the total oxygen-containing offgas.

In the reaction in step c, the reaction mixture is typically contacted with an oxygen-containing gas, wherein the oxygen-containing gas has an oxygen content within the range from 1% to 40% by volume, preferably 5% to 22% by volume, based on the total oxygen-containing gas. Preferably, air can be used as the oxygen-containing gas in step c.

In the reaction in step c, an oxygen-containing offgas is particularly preferably obtained, wherein the oxygen-containing offgas is cooled in at least one step and wherein the oxygen-containing offgas has an oxygen content within the range from 1% to 10% by volume, preferably 1% to 5% by volume, based on total oxygen-containing offgas. In particular, it is possible to achieve condensation and removal of organic components in the offgas through an at least single-stage cooling operation, with the offgas then preferably being able to be recycled into the process.

In a preferred embodiment, a reaction mixture comprising the first reaction product and the second catalyst C2 is used in the reaction in step c, said reaction mixture having a content of polyhydric alcohol(s) of less than 10% by weight, preferably less than 8% by weight, more preferably less than 5% by weight, based on the reaction mixture.

In a preferred embodiment, a reaction mixture comprising the first reaction product and the second catalyst C2 is used in the reaction in step c, said reaction mixture having a content of (meth)acrolein of less than 10% by weight, preferably less than 8% by weight, more preferably less than 5% by weight, based on the reaction mixture.

The reaction of the first reaction product with oxygen in step c is preferably carried out within a temperature range from 0° C. to 120° C., more preferably from 50° C. to 120° C., particularly preferably 60° C. to 100° C. The reaction of the first reaction product with oxygen in step c is preferably carried out within a pressure range from 0.5 to 50 bar (absolute), more preferably from 1 to 50 bar, (absolute), particularly preferably from 2 to 30 bar (absolute). In particular, the reaction in step c is carried out at a temperature and/or a pressure within the ranges mentioned above.

In a preferred embodiment, the reaction of the first reaction product comprising at least a cyclic acetal with oxygen in step c can be carried out in the presence of a solvent. A solvent typically selected from linear or cyclic alkanes (e.g. hexane, octane, cyclohexane), aromatic hydrocarbons (e.g. toluene, benzene), halogenated hydrocarbons (e.g. chloroform, carbon tetrachloride, hexachloroethane, hexachlorocyclohexane, chlorobenzene), alcohols (e.g. methanol, ethanol, n-butanol, tert-butanol), ethers (e.g. diisopropyl ether, tetrahydrofuran, 1,3-dioxane), esters (e.g. methyl acetate, ethyl acetate), nitriles (e.g. acetonitrile) or mixtures thereof can be used. Preferred esters are C₁-C₂₀ alkyl esters, preferably C₁-C₁₀ alkyl esters, of aliphatic and aromatic C₁-C₂₀ carboxylic acids, e.g. of formic acid, acetic acid, propanoic acid, butanoic acid, pentanoic acid, hexanoic acid and benzoic acid. The reaction in step c is typically carried out in a reaction mixture containing 1% to 90% by weight, preferably 5% to 50% by weight, based on the entire reaction mixture, of at least one solvent.

The oxidative reaction of the cyclic acetal in step c typically is incomplete. The process according to the invention preferably includes a workup step d, wherein the cyclic acetal is at least partially removed from the second reaction product comprising the hydroxyalkyl (meth)acrylate ester and optionally recycled into the reaction in step c. For example, the removal of the cyclic acetal from the second reaction product can be carried out in one or more distillation steps (distillation columns).

The process according to the invention can optionally comprise further steps for the workup and/or purification of the second reaction product comprising at least one hydroxyalkyl (meth)acrylate ester. The further workup and/or purification steps typically comprise distillation steps and/or extraction steps and/or crystallization steps.

In a preferred embodiment, a crude product comprising hydroxyalkyl (meth)acrylate esters from the second reaction product is obtained in at least one distillation step (e.g. column 16). In this distillation step, unreacted acetal and optionally solvent can typically be removed from the second reaction product and optionally recycled into the oxidation after step c).

In a preferred embodiment, the reaction in step a is carried out within a temperature range from −50° C. to 100° C., preferably −10° C. to 30° C., more preferably 0° C. to 20° C., and the reaction in step c within a temperature range from 0° C. to 120° C., preferably 50° C. to 120° C., more preferably 60° C. to 100° C., wherein the reaction in step a is carried out at a temperature that is at least 15° C. lower than the temperature in the reaction in step c.

In a preferred embodiment, the reaction in step a is carried out within a pressure range from 0.5 to 10 bar (absolute), preferably 1 to 5 bar (absolute), and the reaction in step c within a pressure range from 1 to 50 bar (absolute), preferably 2 to 30 bar (absolute), wherein the reaction in step c is carried out at a pressure that is at least 0.1 bar absolute, preferably at least 0.5 bar absolute, higher than the pressure in the reaction in step a.

The process according to the invention can typically include the addition of one or more stabilizers, for example selected from polymerization inhibitors, radical scavengers and antioxidants, in particular stabilizers as described in EP-B 1 125 919. For example, the stabilizer may be selected from phenol, substituted phenols (e.g. 4-methoxyphenol), hydroquinone, alkyl-substituted hydroquinones (e.g. methylhydroquinone, tert-butylhydroquinone, 2,6-di-tert-butyl-parahydroquinone, 2,5-di-tert-butylhydroquinone); saturated hydroxyalkyl carboxylates (e.g. hydroxyethyl acetate, hydroxyethyl propionate, hydroxyethyl isobutyrate, hydroxypropyl acetate) and N-oxyl compounds (e.g. piperidine oxyl compounds such as 4-hydroxy-2,2,6,6-tetramethylpiperidine-1-oxyl).

The addition of a stabilizer as described above can typically take place in one or more of steps a, b and/or c. Likewise, the addition of a stabilizer as described above can take place before or after one of steps a, b and/or c. Typically, one or more stabilizers can be added to the first reaction product. The addition of the stabilizer can for example take place after the reaction in step a, for example in step b.

### DESCRIPTION OF THE FIGURES

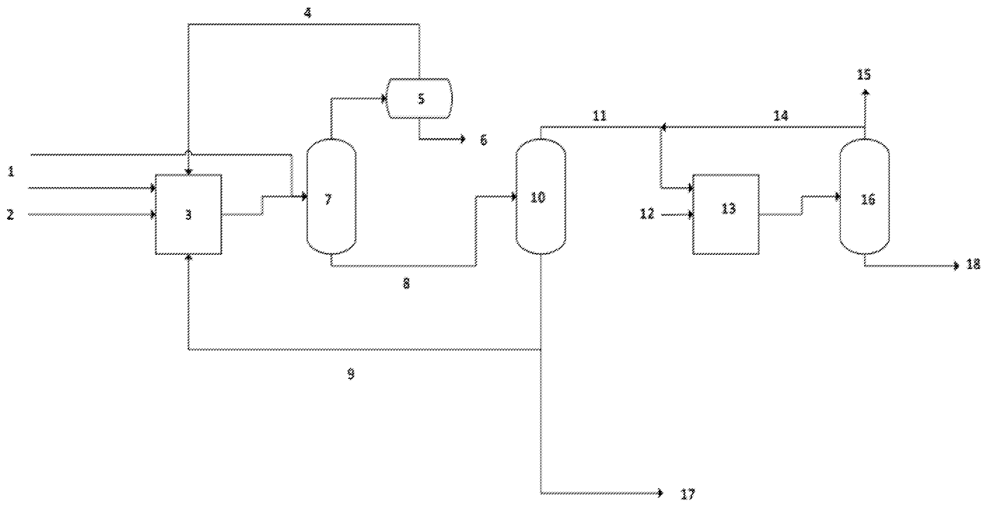

FIG. 1 shows by way of example a possible schematic flow diagram of the process according to the invention. The designations here are defined as follows:
1 (Meth)acrolein stream from 3 or 7
2 Dialcohol stream
3 Reactor, first step, formation of the cyclic acetal
4 Optional recycling stream ((meth)acrolein)
5 Decanter
6 Removal of water
7 Distillation column, separation of (meth)acrolein and water
8 Crude stream of first reaction product (cyclic acetal)
9 Optional recycling stream (dialcohol)
10 Distillation column, separation of acetal from dialcohol and high boilers
11 Acetal stream from second reaction step, optional addition of stabilizer and solvent to second reaction step possible here
12 Metering-in of air for oxidation of the acetal
13 Reactor, second step, formation of the hydroxyalkyl (meth)acrylate ester
14 Optional recycling stream (acetal and/or solvent)
15 Offgas line
16 Distillation column, separation of acetal and/or solvent
17 Discharge of high boilers from the first reaction step
18 Crude stream of second reaction product (hydroxyalkyl (meth)acrylate ester)

### EXPERIMENTAL SECTION

### Example 1a—Continuous Production of the Cyclic Acetal (2-isopropenyl-1,3-dioxolane) Based on Methacrolein and Ethylene Glycol/Methacrolein on a Distillation Column

A jacketed loop reactor containing a 5 kg quantity of active catalyst and having a total volume of 25 L was used. The catalyst (C1) used was a sulfonic acid resin from Lanxess (K2431). The reactor was controlled via the jacket (operated with Aral antifreeze coolant) such that the internal temperature was 2-3° C. The reactor was connected to a distillation column (DN 150 mm, height 6 m) packed with Sulzer DX packing (HETP 60 mm, ˜16.6 theoretical plates per metre packing height).

Ethylene glycol (EG) (15 kg/h, 242 mol/h), to which 100 ppm of TEMPOL (4-hydroxy-2,2,6,6-tetramethylpiperdine-1-oxyl) was added, was metered directly into the reactor (3), whereas the methacrolein (MAL) together with the outflow from the reactor was fed onto a distillation column (7). This resulted in both the methacrolein (MAL) and the reaction product being dewatered, which was advantageous for reaction control. The hetero-azeotrope of methacrolein and water collected at the head of the column, which on cooling separates into two phases. The methacrolein was fed via a decanter (5) back into the reactor (3). The amount of fresh methacrolein was adjusted so that the amount of methacrolein fed into the reactor was 17.25 kg (246 mol) per hour. The molar ratio of methacrolein to ethylene glycol was thus 1.02 and the LHSV (liquid hourly space velocity) was 6.4 ((kg MAL+kg EG)/(kg cat.*hr)).

The contents of the loop reactor were circulated via a pump such that the internal circulation ensured thorough mixing of the reactants; at low circulation flow rates, the formation of two phases was observed. To improve mixing, a static mixer was installed in front of the catalyst bed. The dwell time in the reactor was nearly 45 minutes and the reaction mixture in the reactor outflow had a composition of 40% by weight of methacrolein, 34% by weight of ethylene glycol, 21% by weight of acetal, 3% by weight of water and 2% by weight of secondary components. The secondary components are in particular high-boiling products of the addition of glycol or water to the acetal.

The reactor was started up over a period of 3 hours and was then operated with these parameters continuously and stably for 12 hours. Methacrolein conversion was 26% and selectivity in respect of the acetal was 92%.

### Example 1b—Continuous Removal of Methacrolein and Water from the Product Mixture from Example 1a

The reaction outflow from Example 1a (32.25 kg/h) was mixed with fresh methacrolein and fed into a distillation column (7) (DN 150 mm, height 6 m) packed with Sulzer DX packing (HETP 60 mm, ˜16.6 theoretical plates per metre packing height). The column was operated at a pressure of 90 mbar, a bottoms temperature of 90° C., a distillate temperature of 5° C. and a reflux ratio of 1. Connected at the head of the column was a decanter (5), by means of which the resulting hetero-azeotrope of methacrolein and water (98.8% by weight of MAL and 1.2% by weight of water) was separated.

The aqueous phase in the decanter consisted of 93.9% by weight of water and 6.1% by weight of methacrolein. The aqueous phase was stripped from time to time, yielding a methacrolein-free aqueous bottoms. This bottoms can be treated biologically or incinerated. The organic phase of the decanter (5) was recycled into the reaction (reactor 3).

The bottoms of the distillation column (7) (18.4 kg/h) consisted of the cyclic acetal (37% by weight), ethylene glycol (60% by weight) and the high-boiling by-products mentioned under 1a (3% by weight).

The fresh methacrolein was loaded onto the distillation column (7) in a manner that ensured almost total removal of the methacrolein and water azeotrope from the bottoms of column 7. This achieved depletion of the methacrolein in the bottoms discharge (from 7) to a level of below 1000 ppm.

### Example 1c—Continuous Production of the Cyclic Acetal (2-isopropenyl-1,3-dioxolane) Based on Methacrolein and Ethylene Glycol/Methacrolein in the Reactor

A jacketed loop reactor containing a 5 kg quantity of active catalyst and having a total volume of 25 L was used. The catalyst (C1) used was a sulfonic acid resin from Lanxess (K2431). The reactor was controlled via the jacket (operated with Aral antifreeze coolant) such that the internal temperature was 2-3° C.

The ethylene glycol (15 kg/h, 242 mol/h) and the methacrolein (17.25 kg/h, 246 mol/h) were metered into the reactor (3) with 100 ppm of TEMPOL. The molar ratio of methacrolein to ethylene glycol was thus 1.02 and the LHSV was 6.4 ((kg MAL+kg EG)/(kg cat.*hr)). The contents of the loop reactor were circulated via a pump such that the internal circulation ensured thorough mixing of the reactants; at low circulation flow rates, the formation of two phases was observed. To improve mixing, a static mixer was installed in front of the catalyst bed.

The dwell time in the reactor (3) was nearly 45 minutes and the reaction mixture at the outflow had a composition of 40% by weight of methacrolein, 34% by weight of ethylene glycol, 21% by weight of acetal, 3% by weight of water and 2% by weight of secondary components. The secondary components are in particular high-boiling products of the addition of glycol or water to the acetal.

The reactor was started up over a period of 3 hours and was then operated with these parameters continuously and stably for 12 hours. Methacrolein conversion was thus 26% and selectivity in respect of the acetal was 92%.

### Example 1d—Continuous Removal of Methacrolein and Water from the Product Mixture from Example 1c

The reaction outflow from Example 1c (32.25 kg/h) was fed into a distillation column (7) (DN 150 mm, height 6 m) packed with Sulzer DX packing (HETP 60 mm, ˜16.6 theoretical plates per metre packing height). The column was operated at a pressure of 85 mbar, a bottoms temperature of 88° C., a distillate temperature of 5° C. and a reflux ratio of 2.5. Connected at the head of the column was a decanter (5), by means of which the resulting hetero-azeotrope of methacrolein and water (98.8% by weight of MAL and 1.2% by weight of water) was separated.

The aqueous phase in the decanter consists of 93.5% by weight of water, 6.1% by weight of methacrolein and 0.4% by weight of acetal. The aqueous phase was stripped from time to time, yielding a methacrolein-free aqueous bottoms. This bottoms can be treated biologically or incinerated. The organic phase of the decanter was recycled into the reaction (3) and contained 2.3% by weight of acetal here.

The bottoms of the distillation column (7) (18.4 kg/h) consisted of the cyclic acetal (36% by weight), ethylene glycol (61% by weight) and the high-boiling by-products mentioned under 1a (3% by weight). Although depletion of methacrolein and water in the bottoms to below 1000 ppm was achieved, there was some loss of acetal in the decanter (5).

### Example 1e—Batchwise Production of the Cyclic Acetal (2-isopropenyl-1,3-dioxolane) Based on Methacrolein and Ethylene Glycol/with the Aid of an Inert Azeotropic Entrainer

A glass apparatus fitted with a Dean-Stark apparatus was charged with 315 g of methacrolein (4.5 mol), 279 g of ethylene glycol (4.5 mol), 500 g of hexane and 5.2 g of phosphoric acid (1 mol %). The reaction mixture was stabilized with 0.4 g of TEMPOL and 0.4 g of 4-methoxyphenol in each case. The mixture was heated to 80° C. for 6 hours, resulting in the removal of the water liberated in the reaction by the hexane entrainer. In the separation part of the Dean-Stark apparatus, a water-rich fraction was additionally collected, and the condensed organic fraction consisting of hexane and methacrolein was continuously recycled into the reaction part of the apparatus. After 6 hours the reaction was stopped.

Methacrolein conversion was approx. 70% and selectivity was approx. 48%. Not long after the start of the reaction, a dark-coloured turbidity developed in the reaction vessel at the phase boundary between methacrolein/hexane and ethylene glycol, which increased as the reaction progressed. The turbidity here was due in particular to high-boiling polymers formed from methacrolein and glycol and to products of the addition of glycol to the 4-position of methacrolein or the acetal thereof. Increasing the amount of catalyst or scale-up of the reaction to a larger scale accelerated the formation of these high boilers.

The acetal yields obtained with this methodology were essentially unsatisfactory, particularly with regard to space-time yield. The principle of water removal by means of an entrainer and by means of removal of a methacrolein-water azeotrope is however demonstrated.

### Example 2—Synthesis of the Catalyst (C2) for the Oxidation of 2-Isopropenyl-1,3-Dioxolane to Hydroxyethyl Methacrylate (HEMA) Example 2a

0.90 g of bismuth pentahydrate and 0.36 g of telluric acid were suspended, with stirring, in a glass apparatus. HNO₃ (60%) was added dropwise until everything had dissolved and the formation of unstable suboxides was prevented. 20.0 g of palladium on alumina (5% by weight Pd) was added and the suspension heated to 60° C. On reaching this temperature, the mixture was stirred for one hour. To this was added dropwise 10.0 g of a hydrazine monohydrate solution. The suspension was heated to 90° C. and stirred for another hour. After cooling to room temperature, the black solid was filtered off and washed with four 100 mL portions of distilled water. The conductivity of the liquid from the last wash was less than 100 μS/cm, which showed that the dopants had been taken up quasi-quantitatively.

The solid was dried at 105° C. for 10 h, affording the final catalyst. The stoichiometry was Pd1.00Bi0.20Te0.17@Al2O3.

### Example 2b

The synthesis was carried out in analogous manner to Example 2a, without addition of telluric acid.

### Example 2c

The synthesis was carried out in analogous manner to Example 2a, without addition of bismuth nitrate pentahydrate.

### Example 2d

The synthesis was carried out in analogous manner to Example 2a, with addition of twice the amount of telluric acid.

### Example 2e

The synthesis was carried out in analogous manner to Example 2a, with addition of twice the amount of bismuth nitrate pentahydrate.

### Example 2f

The synthesis was carried out in analogous manner to Example 2a, with addition of twice the amount of bismuth nitrate pentahydrate and telluric acid.

### Example 3—Oxidation of 2-isopropenyl-1,3-dioxolane to Hydroxyethyl Methacrylate (HEMA)

### Example 3a

Into a 130 mL steel autoclave with stirrer unit was weighed 400 mg of catalyst (C2) from Example 2a and a 25% by weight solution of 2-isopropenyl-1,3-dioxolane in toluene stabilized with 200 ppm of TEMPOL. The autoclave was closed, pressurized to 37 bar with 7% oxygen in nitrogen (0.6 equivalents of oxygen per equivalent of acetal) and placed in a pre-tempered oil bath at 70° C. The reaction was stirred for 4 h and then stopped by cooling with dry ice. The pressure in the autoclave was carefully released and the reaction mixture was analysed by gas chromatography (GC).

Conversion was 54% and selectivity in respect of 2-hydroxyethyl methacrylate was 84%. This corresponds to a space-time yield of 3.8 mol HEMA/kg catalyst per hour. Ethylene dimethacrylate could not be detected by gas chromatography (GC).

### Example 3b

The oxidation was carried out as in Example 3a, but using ethyl acetate as solvent. After a reaction time of 2 hours, conversion was 91%, selectivity was 88% and the space-time yield was 19.6 mol HEMA/kg catalyst per hour. Ethylene dimethacrylate could not be detected by gas chromatography.

### Example 3c

The oxidation was carried out as in Example 3a, but with the catalyst from Example 2b. Conversion was 12% and selectivity was 79%.

### Example 3d

The oxidation was carried out as in Example 3a, but with the catalyst from Example 2c. Conversion was 73%, selectivity was 78% and the space-time yield was 7.1 mol HEMA/kg catalyst per hour.

### Example 3e

The oxidation was carried out as in Example 3a, but with the catalyst from Example 2d. Conversion was 20%, selectivity was 84% and the space-time yield was 2.1 mol HEMA/kg catalyst per hour.

### Example 3f

The oxidation was carried out as in Example 3a, but with the catalyst from Example 2e. Conversion was 83%, selectivity was 59% and the space-time yield was 6.1 mol HEMA/kg catalyst per hour.

### Example 3g

The oxidation was carried out as in Example 3a, but with the catalyst from Example 2f. Conversion was 59%, selectivity was 71% and the space-time yield was 5.3 mol HEMA/kg catalyst per hour.

### Example 3h

The oxidation was carried out as in Example 3a, but using ethylene glycol as solvent. After a reaction time of 2 hours, conversion was 99%, selectivity was 55% and the space-time yield was 7.06 mol HEMA/kg catalyst per hour.

The main side reaction observed was hydrogenation of 2-hydroxyethyl methacrylate. Selectivity in this reaction was 30%. The example demonstrates that, for reaction control and for the achievement of high selectivities and yields, it is advantageous to monitor and reduce the concentration of the alcohol (reactant in the first step), since the side reaction to the undesired, hydrogenated by-product can otherwise increase.

### Example 3i

The oxidation was carried out as in Example 3a, but with the reaction carried out at atmospheric pressure and with the gas amount chosen such that there was a continued excess of oxygen. After a reaction time of 4 hours, almost no conversion was present. The reaction time was extended to 48 hours, wherein conversion of approx. 50% was observed, with selectivity of 83%.

This demonstrates that reaction at lower pressures, although possible, is economically unviable.

### Example 3j

The oxidation was carried out as in Example 3a, but with the reaction carried out at 50° C. After a reaction time of 4 hours, conversion was 29% and selectivity was 83%. On lowering the temperature, an almost linear decrease in reaction rate was observed.

This demonstrates that reaction at low temperatures, although possible, is economically unviable.

1: A process for producing hydroxyalkyl (meth)acrylate esters, the process comprising:
a) reacting (meth)acrolein with at least one polyhydric alcohol in the presence of a first catalyst C1, wherein a first reaction product comprising at least one cyclic acetal is obtained;
b) at least partially removing water from the first reaction product; and
c) reacting the first reaction product with oxygen in the presence of a second catalyst C2, wherein a second reaction product comprising at least one hydroxyalkyl (meth)acrylate ester is obtained.

2: The process according to claim 1, wherein the process is a continuous process for producing hydroxyalkyl (meth)acrylate esters.

3: The process according to claim 1, wherein the reaction in a) is carried out in the presence of at least one acidic compound as the first catalyst C1, selected from the group consisting of Brønsted acids and Lewis acids,
wherein the first catalyst C1 is present in heterogeneous or homogeneous form and comprises at least one acidic group that has a pKa of less than or equal to 5.

4: The process according to claim 1, wherein the at least one fir catalyst C1 is selected from the group consisting of phosphoric acid, sulfuric acid, sulfonic acid, carboxylic acid, and an ion-exchange resin containing at least one acidic group selected from the group consisting of sulfonic acids and carboxylic acids.

5: The process according to claim 1, wherein the reaction in a) is carried out with a molar ratio of (meth)acrolein to polyhydric alcohol(s) within a range from 1:50 to 50:1.

6: The process according to claim 1, wherein, in b), unreacted (meth)acrolein and/or unreacted polyhydric alcohol are removed from the first reaction product and recycled into the reaction in a).

7: The process according to claim 1, wherein, in b), in a first separation, (meth)acrolein and at least some of the water are removed from the first reaction product.

8: The process according to claim 1, wherein, in b), in at least two separations, water, unreacted (meth)acrolein and unreacted polyhydric alcohol are removed from the first reaction product comprising at least one cyclic acetal.

9: The process according to claim 1, wherein the reaction in c) is carried out in the presence of a metal-containing and/or metalloid-containing, heterogeneous catalyst system as the second catalyst C2.

10: The process according to claim 1, wherein the at least one second catalyst C2 is a heterogeneous catalyst comprising one or more support materials and one or more active components,
wherein the one or more support materials are selected from the group consisting of activated charcoal, silicon dioxide, aluminium oxide, titanium dioxide, alkali metal oxide, alkaline earth metal oxide, and a mixture thereof, and
wherein the one or more active component comprise at least one element selected from the group consisting of palladium, platinum, iridium, rhodium, ruthenium, gold, cobalt, nickel, zinc, copper, iron, selenium, tellurium, arsenic, antimony, bismuth, germanium, tin, and lead,
wherein the at least one element is present in elemental form, as an alloy, or in the form of a compound thereof in any desired oxidation state.

11: The process according to claim 1, wherein the at least one second catalyst C2 is a heterogeneous catalyst comprising silicon dioxide and/or aluminium oxide as a support material, and comprising as an active component at least one element selected from the group consisting of palladium, bismuth, and tellurium,
wherein the at least one element can be present in elemental form, as an alloy, or in the form of a compound thereof in any desired oxidation state.

12: The process according to claim 1, wherein the at least one second catalyst C2 is a heterogeneous catalyst comprising silicon dioxide and/or aluminium oxide as a support material, and an active component, wherein the active component is palladium and at least one further element selected from the group consisting of selenium, tellurium, and bismuth,
wherein the at least one further element can be present in elemental form, as an alloy, or in the form of an oxide thereof in any desired oxidation state.

13: The process according to claim 1, wherein, in the reaction in c), a reaction mixture comprising the first reaction product and the second catalyst C2 is contacted with an oxygen-containing gas,
wherein an oxygen-containing offgas is obtained in c), wherein the oxygen-containing offgas is cooled at least once, and wherein the oxygen-containing offgas has an oxygen content within the range from 1% to 10% by volume based on the total oxygen-containing offgas.

14: The process according to claim 1, wherein a reaction mixture comprising the first reaction product and the second catalyst C2 is used in the reaction in c), said reaction mixture having a content of polyhydric alcohol(s) of less than 10% by weight based on the reaction mixture in c).

15: The process according to claim 1, wherein the reaction in a) is carried out within a temperature range from −50° C. to 100° C. and the reaction in c) is carried out within a temperature range from 0° C. to 120° C., and
wherein the reaction in a) is carried out at a temperature that is at least 15° C. lower than the temperature in the reaction in c).

16: The process according to claim 1, wherein the reaction in a) is carried out within a pressure range from 0.5 to 10 bar and the reaction in c) is carried out within a pressure range from 1 to 50 bar, and
wherein the reaction in c) is carried out at a pressure that is at least 0.1 bar higher than the pressure in the reaction in a).

17: The process according to claim 5, wherein the reaction in a) is carried out with a molar ratio of (meth)acrolein to polyhydric alcohol(s) within a range from 1:3 to 3:1.
